(19) United States
(12) Patent Application Publication
(10) Pub. No.: US 2023/0210095 A1
(43) Pub. Date: Jul. 6, 2023

(54) WATER CIRCULATION AND AERATION SYSTEM FOR AQUACULTURE FACILITY, RELATED FACILITY, METHODS AND USE

(71) Applicant: LUONNONVARAKESKUS, Helsinki (FI)

(72) Inventor: Tapio KIURU, Laukaa (FI)

(21) Appl. No.: 17/927,666

(22) PCT Filed: May 26, 2021
(86) PCT No.: PCT/FI2021/050375

§ 371 Date: Nov. 23, 2022

(30) Foreign Application Priority Data
May 26, 2020 (FI) 20205531

(51) Int. Cl. A01K 63/04

(52) U.S. Cl. CPC A01K 63/042; A01K 63/047

(57) ABSTRACT

A circulation and aeration system 10 for an aquaculture facility including an elongated reservoir for cultivating aquatic species is provided. The system includes a number of vertical manifolds distributed along the length of the reservoir through an interconnecting piping and arranged to generate a number of adjacent rotating liquid flow patterns by injecting aerated water into the reservoir via exhaust ports, wherein each the pattern forms an individual rotating flow cell, and wherein the system further includes at least one airlift pump configured to supply the aerated water into the manifolds. Related facility, methods and uses are further provided.

### CROSS-REFERENCE TO RELATED APPLICATION(S)

This application is the U.S. national phase of International Application No. PCT/FI2021/050375 filed May 26, 2021, which designated the U.S. and claims priority to FI 20205531 filed May 26, 2020, the entire contents of each of which are hereby incorporated by reference.

### BACKGROUND OF THE INVENTION

### Field of the Invention

Generally, invention relates to water treatment systems and methods exploited in rearing of aquatic species. In particular, the present invention concerns a water circulation and aeration system for a raceway aquaculture facility, in which system circular flow patterns are generated by airlift pump(s). The invention further concerns an aquaculture facility, related methods and uses.

### Description of the Related Art

Aquaculture, generally referred to as farming of aquatic organisms, such as fish and shellfish, under controlled conditions, is a fast-growing agricultural sector that allows for harvesting seafood for human and animal consumption. By means of aquaculture a variety of aquatic species can be produced in a cost-effective manner and with predictable yields regardless of external conditions, such as weather and/or population fluctuations.

In recent decades, fish harvesting in aquaculture facilities with complete or partial water recirculation has gained popularity and profitability over traditional rearing of fish in open ponds. Recirculation aquaculture systems (RAS) vary in design, according to production goals and geographical locations. Common practices include using raceway systems configured as rectangular tanks with baffles or a series of tanks, or using rearing facilities comprising a series of round-shaped ponds or tanks, in where circular water current is established around a central drain.

A mixed cell raceway (MCR) system proposed in the 2000s (Watten et al) allows to establish rotating water currents in existing rectangular raceway or pond systems. In conventional MCR system, a number of so called cells in established in a rectangular vessel by virtue of creating adjacent circular water currents that enable mixing liquid within each cell and between adjacent cells. Implemented without solid boundaries between the cells, the MCR system allows for mixing the active volumes of each cell.

Conventional MCR systems are constructed as stationary, permanent facilities that are still in need of improvements. Particular limiting factors include inefficiency in adapting a cultivation facility to conditions of seasonal rainfalls or dryness; structural rigidity in scaling up (or -down) the production capacity and, as a consequence, inability to address the needs of both industrial manufacturers and private farmers.

Moreover, in existing MCR units, circular water currents are generated cell-wise by means of conventional water pumps. In order to address the issues of aeration and carbon dioxide removal from water, the facility must include additional aeration and oxygenation appliances, such as trickling towers and/or diffuser aerators, oxygen generator(s) and devises like oxygen cones to dissolve oxygen into water. Also other water treatment units like bioreactors are typically included into the MCR systems as external units.

### SUMMARY OF THE INVENTION

An objective of the present invention is to at least alleviate each of the problems arising from the limitations and disadvantages of the related art. The objective is achieved by various embodiments of a water circulation and aeration system for an aquaculture facility, related facility, methods and uses.

In an aspect, a water circulation and aeration system for an aquaculture facility is provided.

In embodiment, the water circulation and aeration system for an aquaculture facility comprising an elongated reservoir for cultivating aquatic species is provided, said system comprises a number of vertical manifolds distributed along the length of the reservoir through an interconnecting piping and arranged to generate a number of adjacent rotating liquid flow patterns by injecting aerated water into said reservoir via exhaust ports, wherein each said pattern forms an individual rotating flow cell, and wherein the system further comprises at least one airlift pump configured to aerate water and to supply aerated water into the manifolds.

In embodiment, the manifolds are arranged in the system to generate rotating flow patterns independently for each individual flow cell.

In embodiment, the system is configured to establish the flow patterns cell-wise in an absence of physical boundaries between the cells.

In embodiment, the adjacent cells are established by the counter flow patterns.

In embodiment, the system further comprises a foam collector device connected to the airlift pump conduit.

In another aspect, an aquaculture facility is provided.

The facility comprises an elongated reservoir configured to receive liquid to accommodate aquatic species and to establish a cultivation area, and a water circulation and aeration system according to the previous embodiments.

In embodiment, the facility comprises the reservoir formed by an enclosed environment on land. In embodiment, the facility comprises the reservoir provided as an elongated container, such as an ISO standard shipping tank.

In embodiment, the facility further comprises a water treatment area separated from the cultivation area by a partition. Said partition enables water exchange between the cultivation area and the water treatment area.

In embodiment, the partition is configured movable along the length of the reservoir, and wherein by moving said partition the volume of the water treatment area and the volume of the cultivation area is regulated.

In embodiment, said water treatment area is a compartment filled with bed material configured for purification and/or treatment of liquid arriving thereto from the cultivation area. In embodiment, the water treatment area is configured for nitrification reactions.

In embodiment, the facility further comprises a wastewater drain system comprising a number of vertical manifolds, each said manifold being arranged substantially in the center of a corresponding rotating flow cell, said manifolds being connected at their upper ends through an interconnecting piping configured to withdraw wastewater outside the facility.

In some configurations, the facility further comprises means for supplying hydrogen peroxide into the cultivation reservoir, in (semi)continuous manner or in single injection(s).

In embodiment, the facility is configured as a recirculation aquaculture facility, such as a Recirculation Aquaculture System (RAS) or as a Partial Reuse Aquaculture System (PRAS).

In an aspect, a modular plant for cultivating aquatic species is provided. The plant comprises a number of modules, wherein each said module is configured as an aquaculture facility according to described embodiments and wherein said modules are configured to operate, individually or collectively, in any one of a flow-through mode, a Recirculation Aquaculture System (RAS) mode or a Partial Reuse Aquaculture System mode, and to switch between said modes as required.

In a further aspect, a method for water circulation and aeration in an aquaculture facility is provided7.

In a further aspect, a method for treating water in an aquaculture facility is provided.

In still further aspect, use of the aquaculture facility according to some previous embodiments in cultivating aquatic species, such as fish and/or crustaceans, is provided.

The utility of the present invention arises from a variety of reasons depending on each particular embodiment thereof.

The innovative solution disclosed hereby exploits airlift pumps for improved aeration and removal of carbon dioxide from the cultivation reservoir. Hence, one of the primary advantages of using airlifts instead of conventional water pumps is enabling efficient gas exchange.

In farming of aquatic species, carbon dioxide produced by said species, e.g. fish, must be vented out from the pond. At the same time, aeration, defined as a process of transfer / addition of oxygen into water, is a crucial process in successful cultivation of aquatic species. Oxygen is a relatively rare component in water (10 ppm is considered as high concentration; typical recirculation aquaculture system operating with an oxygen level of 5-8 ppm). Oxygen is consumed by both cultivated species and by bacteria naturally occurring within the cultivation facility. Therefore, under high loads, an exemplary RAS facility must be capable of replacing all oxygen in the system every 20-60 minutes. In an absence of continuous aeration the entire cultivated population can be lost.

Standard centrifugal- or axial-flow pumps typically used for water transfer in aquatic farming facilities do not have any added functionalities with regard to aeration and/or removal of undesirable gases (e.g. CO₂). Conventional facilities typically comprise aeration tanks separate from the cultivation tanks. Alternatively, water may be pumped to a significantly higher level, from there water may be trickled through a porous structure, whereupon aeration occurs.

Thus, in conventional aquaculture facilities, pumping and aeration processes require utilization of separate equipment and consume high amounts of energy. On the contrary, in airlift pumps a process of gas exchange shares energy and equipment with the process of water transfer / pumping. By means of airlifts, the most essential in the aquaculture processes, such as flow intake, addition of oxygen and removal of carbon dioxide, can be integrated within an individual cultivation facility.

Additionally, the solution allows for simultaneous removal of settleable solids through bottom drains and fine solids from the growth liquid by flotation, foam fractionation and/or protein skimming.

The solution further involves a built-in (bio)reactor, whose volume can be flexibly modified with regard to the volume of the rest of the facility used for cultivation and harvesting.

The inventive concept disclosed hereby further provides a modular, plug-and-play solution for an aquaculture facility.

The expression “a number of” refers hereby to any positive integer starting from one (1), e.g. to one, two, or three. The expression “a plurality of” refers hereby to any positive integer starting from two (2), e.g. to two, three, or four.

Different embodiments of the present invention will become apparent by consideration of the detailed description and accompanying drawings.

### BRIEF DESCRIPTION OF THE DRAWINGS

FIG. 1 schematically illustrates a basic concept of an airlift driven circulation and aeration system 10 within an aquaculture facility 100, according to an embodiment.

FIG. 2 schematically illustrates formation of rotating flow patterns in virtual flow cells.

FIG. 3 schematically illustrates the aquaculture facility 100 with a water treatment area, according to an embodiment.

FIG. 4 schematically illustrates the aquaculture facility 100 with a vertical drain system, according to an embodiment.

### DESCRIPTION OF THE PREFERRED EMBODIMENTS

Detailed embodiments of the present invention are disclosed herein with the reference to accompanying drawings. The same reference characters are used throughout the drawings to refer to same members. Following citations are used for the members:
10 – a water circulation and aeration system;
11A, 11B – distribution piping;
12 – exhaust ports;
13 – an airlift pump;
13A, 13B – an air injection line and an air-relief device, accordingly;
14 – drain(s);
15, 15A – a foam collector device and a discharge pipe, accordingly;
16, 16A – water supply line(s) and related valve(s);
17 – an inflow into the airlift pump;
18 – a partition;
20 – a reservoir;
20A, 20B, 20C – rotating flow cells;
21 – a cultivation area;
31 – a water treatment area;
40 – a wastewater drain system;
41A, 41B – distribution piping for the wastewater drain system;
42, 43 – wastewater drain piping: inlet(s) and exhaust end, accordingly;
51 – an air blower / air compressor;
52 – an operation and control section;
53 – a wall between the section 52 and the rest of the reservoir 20;
100 – an aquaculture facility.

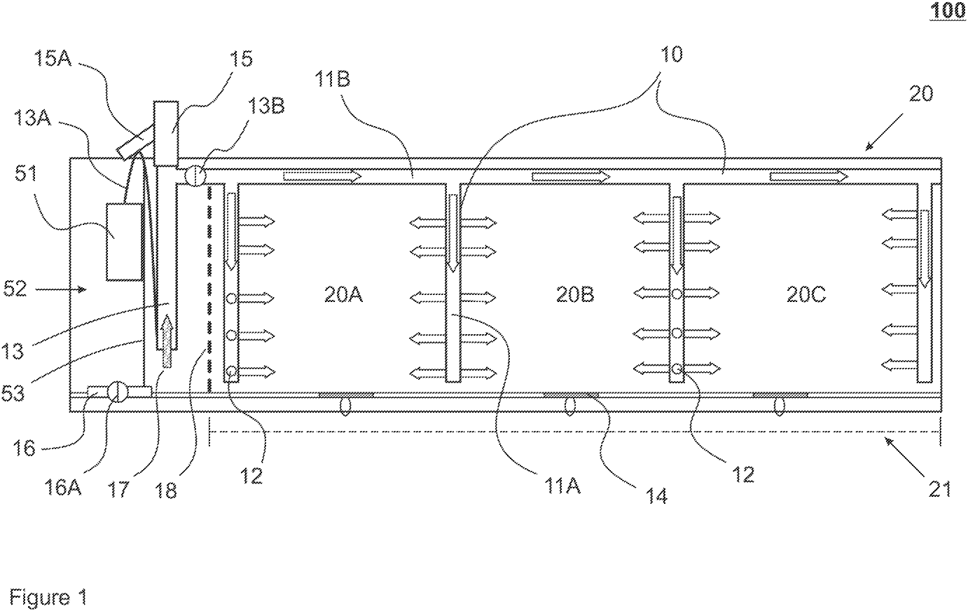

A water circulation and aeration system 10 and an aquaculture facility 100 comprising said system 10 are schematically illustrated on FIG. 1. The facility 100 comprises an elongated reservoir 20 for cultivating aquatic species and the system 10 that enables liquid circulation and aeration in the reservoir.

The facility 100 can be configured as a recirculation aquaculture facility, such as a Recirculation Aquaculture System (RAS) or as a Partial Reuse Aquaculture System (PRAS). The facility 100 may be generally described as a mixed cell raceway.

The reservoir 20 can be provided as an elongated container or tank, such as an ISO standard cargo tank. By way of example, standard basic containers (20′ DC, 40′ DC; wherein DC stands for Dry Cargo) or high cube (dry cargo) containers (40′ HC, 45′ HC) can be utilized. In terms of sizes and dimensions, a standard 20′ DC container has volume about 33 m³ and interior dimensions 6 m × 2.34 m × 2.4 m (length, width, height); a standard 40′ DC container has volume 66.4 m³ and interior dimensions 12 m × 2.34 m × 2.4 m; a standard 40′ HC container has volume 73.5 m³ and interior dimensions 12 m × 2.34 m × 2.7 m; and a standard 45′ HC container has volume 86 m³ and interior dimensions 13.5 m × 2.41 m × 2.7 m. Any other suitable ISO standard container can be utilized. The containers can be closed or open-top containers.

A reservoir housing formed by a container made of aluminum, steel, such as COR-TEN® steel or stainless steel, or plywood, for example, can be further provided with a supporting structure implemented as a metal framework with reinforcement- and/or insulation materials (e.g. insulation foams), to create a “waffle” structure. The container may be further lined with a coating or a liner, optionally being flexible.

By utilizing the ISO shipment container, the facility 100 is rendered mobile, thus enabling or at least facilitating its transportation by conventional means (by trucks, marine vessels, etc.).

The reservoir 20 can be formed by any other elongated, optionally essentially rectangular container or tank. Alternatively, the reservoir 20 can be formed by an enclosed environment on land, such as a pond, a basin, a channel, a ditch, and the like. The enclosed environment may be natural or manmade (artificial). In an event the reservoir 20 is built as the enclosed environment on land, it is assumed that the skilled reader would have no difficulty in understanding that the reservoir 20 contains necessary appliances, such as a draining arrangement, for example, to render it suitable for use as the facility 100. Essentially rectangular shape in vertical and horizontal (longitudinal) cross-sections is preferably maintained.

The facility 100 may be configured as a single reservoir 20 supplied with the water circulation and aeration system 10. By connecting a number of such reservoirs 20 to a common water source and/or a draining system a larger facility referred to as an aquaculture plant may be constructed. The plant may comprise 2-20 reservoirs, for example. All or some reservoirs within the plant can be interconnected. By way of example, pairwise connection can be implemented with a shared water treatment section (the latter described further below). The connection may be permanent or temporary to facilitate a “plug-and-play” construction of the modular aquaculture plant. Once connected reservoirs can be disconnected and separated whenever required. The modular construction is particularly preferred when the plant is implemented with the ISO shipment containers.

Within the facility 100, the system 10 comprises a number of vertical manifolds 11A configured as vertical pipes with a plurality of exhaust ports 12, such as jet ports or jet nozzles, arranged along the length of the pipe 11A. Each manifold 11A may comprise ports pointing one direction or different directions. Vertical manifolds 11A extend from the edges of the reservoir, optionally from above the water level, essentially to the bottom of the reservoir. At their upper ends, manifolds 11A are interconnected by means of an interconnecting piping 11B provided as an essentially horizontal tube extending along internal wall(s) of the reservoir 20. The tube 11B may be positioned above the water level.

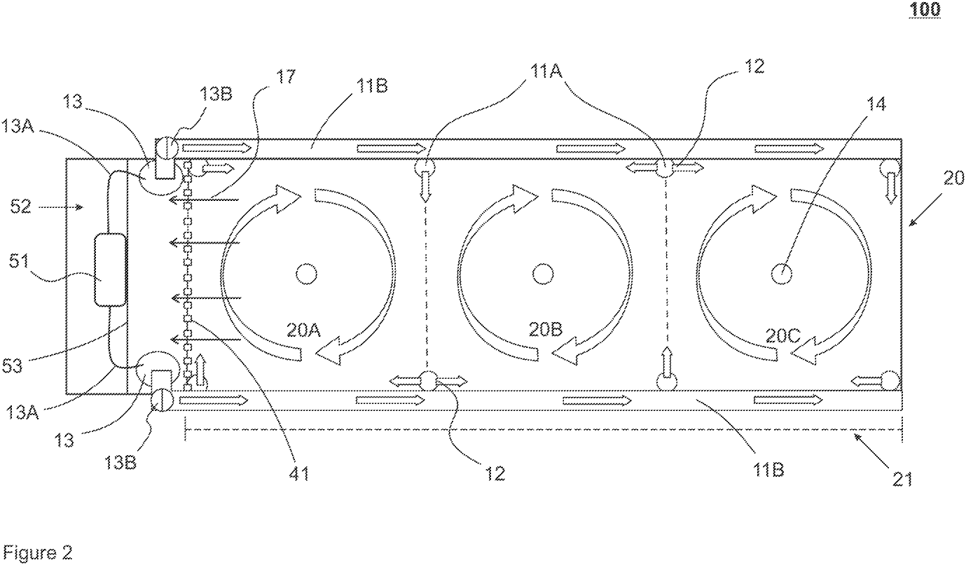

FIG. 2 shows an exemplary layout for the horizontal distribution pipe 11B within the reservoir 20 with regard to vertical manifolds 11A.

In use, the reservoir 20 is filled with water to a predetermined waterline. Through the exhaust ports 12 arranged in the manifolds 11A, water, optionally, a mixture of water and air is directed into the reservoir, whereby a number of adjacent rotating liquid flow patterns are generated. In practice, pairs of vertical manifolds 11A positioned on the opposite walls of the reservoir essentially against one another create a number of virtual “cells” 20A, 20B, 20C, with four manifolds positioned at the corners of each cell. The cell is thus defined as a rectangular area having the manifolds 11A at its corners and a central drain 14 in the middle. The reservoir 20 contains no barriers or baffles between the cells 20A, 20B, 20C; therefore, the cells are referred to as “virtual” cells. Rotating flow patterns are established cell-wise in an absence of physical boundaries between the cells.

Alternatively, mentioned cells can be physically separated from one another by addition of barriers, such as partition walls, between the cells. This may be a scenario, when more than one group of aquatic species is present in the reservoir. By provision of physical barriers between the cells, juvenile fish can be easily separated from adult fish, for example, when kept in the same reservoir.

The facility 100 may be configured for 2-10 adjacent cells. Some stationary implementations may involve more than 10 adjacent cells arranged in one row. In some alternative or additional configurations, the cell rows may be arranged in arrays.

Adjacent cells may have common manifolds 11A or pairs of manifolds. Through the exhaust ports 12, water is injected into the reservoir to establish a rotation around the drain 14 arranged in the bottom of the reservoir 20. Adjacent circular water currents are thus generated cell-wise around the drains 14, along the length of the reservoir 20. Drains 14 are preferably covered with screens.

The ports 12 configured as nozzles, for example, are oriented, along the length (height) of the pipe 11A such, as to achieve the most optimal flow profile. In some instance, direction of injected streams may be essentially tangential with regard to a circumference of the rotating pattern. The ports 12 on the manifolds 11A are thus arranged such the injected streams follow along the circumference of the rotating pattern, at a tangent from a radial direction (the latter defined by a vector between a middle point of a circle and any point at its perimeter / circumference). Flow profiles can be further optimized by arranging and orienting the ports 12, within each vertical manifold 11A, in a predetermined manner, wherein the ports provided in the upper part of each said manifold are oriented to generate essentially tangential fluid flow, whereas the ports provided in the lower part of the manifold are oriented essentially towards the center of the cell.

Optimization of fluidic flow can thus be attained by orienting about a quarter- (¼) to about a half-(½), preferably about one third (⅓), of all ports/nozzles 12 provided on the vertical pipe 11A towards the center of the cell (defined by the drain 14). Preferably, the center-oriented nozzles 12 are disposed in the lower part of the vertical manifold 11A to direct fluidic flow towards the drain 14. Such an arrangement is particularly efficient in removal of sludge from the tank 20, by which virtue nearly self-cleaning functionality can be achieved to minimize (manual) cleaning of the tank.

Individual rotating flow cells 20A, 20B, 20C are thus formed by essentially circular flow patterns or vortices generated upon injecting water into the reservoir via the manifolds 11A.

Rotating flow patterns in the cells 20A, 20B, 20C are generated independently. Thus, flow patterns in adjacent cells may vary in terms of flow velocity and direction, amount of air injected, and other parameters. Independent liquid flow in the adjacent cells may be established by a variety of regulating appliances, such as adjustable-flow valves, flow rate- and/or pressure sensors, rotating nozzles, and the like.

The system 10 may be configured with the adjacent cells established by the counter flow patterns (FIG. 2). Such an arrangement involves alternating cells having liquid currents circulating in opposite directions. Alternatively, the system 10 may be configured with all cells having flow patterns rotating in the same direction (e.g. clockwise or counterclockwise).

In order to supply aerated, oxygen-enriched and essentially void of carbon dioxide water into the reservoir 20 (via distribution piping 11A, 11B), the system 10 comprises at least one airlift pump 13 also referred to as “airlift”. Airlift pump is generally provided as a vertical conduit immersed in the liquid (in the reservoir 20) and connected, at its upper end, optionally above the water level, with the interconnecting piping 11B. An implementation comprising two airlift pumps 13 arranged essentially at the corners of the reservoir 20 (at the same end essentially opposite one another) is illustrated on FIG. 2.

Compressed air is injected at the bottom of the conduit 13 by means of an air blower or an air compressor 51. Air injection line is designated by a reference numeral 13A. By virtue of being less dense than the rest of the liquid (represented with a single-phase water), the air-liquid mixture is caused to propagate upwards through the conduit 13. The resulting buoyancy force of the air bubbles causes a pumping action. From the conduit 13, air-driven water (viz. water driven upwards by virtue of the buoyancy force) is supplied into the interconnecting piping 11B and into the manifolds 11A, accordingly. The conduit 13 may be open at its lower end to recirculate water from the reservoir 20. Additionally or alternatively, the conduit 13 may be constructed to utilize water from an external source (not shown).

The system 10 further comprises at least one an air-relief device 13B installed in the essentially horizontal pipe 11B optionally in conjunction with the airlift pump 13. Each airlift in the system 10 may be provided with at least one separate air-relief device 13B (FIG. 2). The device 13B may be integrated into the conduit 13 to form a part of the airlift pump.

The air-relief device 13B can be configured as a valve, optionally an automated valve, to discharge the most of air trapped in the air-liquid mixture rising along the conduit 13. Such valve has a venting orifice through that air entrained into liquid is released. The air-relief device 13B can be configured as a ventilation aperture or a T-piece to assist release of air accumulating in the pipes. Thus, whereas air is evenly distributed through the (aerated) liquid rising upwards in the vertical airlift 13, in horizontal piping 11B air tends to accumulate at the top side of the pipe (through which liquid propagates towards manifolds 11A). By means of air-relief device(s) efficient control over flow rate and pressure may be enabled.

Liquid, typically water, is aerated and enriched with oxygen during its flow upwards inside the airlift conduit 13. Aeration is accompanied with removal of carbon dioxide. Thereafter, aerated water propagates, optionally past the air-relief valve 13B, into vertical manifolds 11A and, via the exhaust ports 12, into the reservoir 20. Air blown (by means of the airlift 13) aerated water contains dissolved oxygen in an amount normally higher than it is possible to intake via the open surface, for example. Aerated water is supplied to the reservoir 20 by air-driven injection.

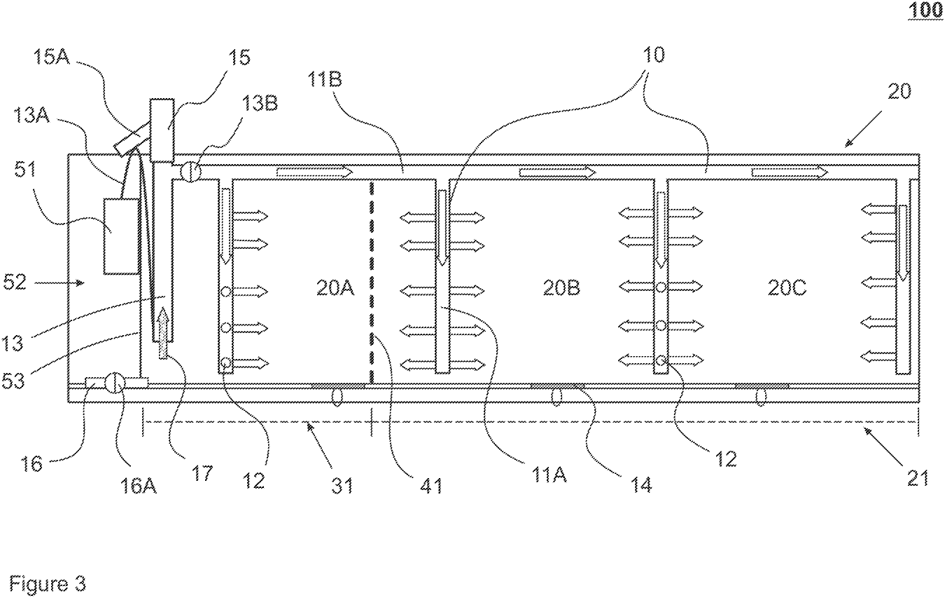

A mixture of water and air propagating through the conduit 13 is indicated on FIGS. 1 and 3 with a reference number 17 (shaded arrow); whereas the aerated water is indicated by empty arrows. While the air-relief valve(s) 13B remove(s) most of air entrained into liquid during its propagation in the conduit 13, liquid/water propagated past said valve(s) still contains added oxygen dissolved therein (in amounts higher than that pertaining to conditions without additional aeration means). Therefore, the air-driven, airlift mediated flow stream directed into the reservoir 20 is referred to as an aerated flow stream. Air-driven injection of aerated water into the reservoir occurs via the exhaust ports 12. Release of entrained air is accompanied by venting off the carbon dioxide; therefore, the air-relief valve(s) additionally provide efficient means for CO₂ removal.

The air blower / air compressor 51 is provided, within the facility 100, in an operation and control section 52 separated from the rest of the reservoir 20 by a water impermeable wall 53. The section 52 is preferably disposed at the end of the facility 100 adjacent to the airlift pump 13. Control over the system 10 and the facility 100 may be fully or partly automated.

In an event the reservoir 20 is provided in the ISO standard shipment container, as described hereinabove, it may be advantageous that the operation and control section 52 is arranged next to the doors of the container to facilitate maintenance works.

In some instances, the facility may incorporate a number of airlift pumps 13 provided at the opposite ends of the reservoir 20 (not shown). This may be a scenario with a stationary facility involving more than 10 adjacent cells. In such an event, the operating sections 52 may be provided at the opposite ends of the facility. Airlifts 13 may be further incorporated into each cell 20A, 20B, 20C, etc. (one or two per cell) or into each vertical manifold 11A. In the latter configuration, all vertical manifolds 11A in the system 10 / the facility 100 may be replaced with airlift conduits 13.

The airlift pump(s) 13 aerate water in the reservoir 20 and account(s) for removal of carbon dioxide therefrom. The airlift pump may be further used to suspend nutrients into the reservoir 20.

The airlift pump conduit 13 may be additionally equipped with a foam / froth collector device 15 provided at the upper end of said conduit. The airlift pump 13 and the device 15 collect and remove (via a discharge pipe 15A) fine solids from the reservoir 20 by flotation, foam fractionation and/or protein skimming.

Water is supplied into the reservoir 20 through a water supply pipe or pipes 16. Water supply is regulated by appropriate valves 16A, such as ball valves (e.g. 15-75 mm ball valves) or any other suitable appliances.

Reference is made to FIGS. 1 and 3 illustrating the aquaculture facility 100 comprising a cultivation area 21 and a water treatment area 31. The cultivation area 21 is established by the entire reservoir 20 or a part thereof configured to receive liquid to accommodate aquatic species for rearing.

The water treatment area 31 is a compartment filled with bed material configured for purification and treatment of liquid arriving thereto from the cultivation area 21. Said bed material may be provided as granules, pellets or fine particulate suitable for mechanical purification, e.g. by filtration, of inflowing liquid and, additionally or alternatively, for (bio)chemical and/or biological treatment of said liquid. The bed material may be configured as a nitrification (bio)reactor for example.

The water treatment area 31 is separated from the cultivation area 21 by a partition 18. The partition 18 can be provided as a screen, a grate, a web or a mesh made of material that enables liquid / water exchange between the cultivation area 21 and the water treatment area 31 and that prevents the bed material from penetrating into the cultivation area 21.

In some configurations, the partition 18 is movable along the length of the reservoir 20. By providing the partition 18 as an element movable along the reservoir 20, the volume of the water treatment area 31 and the volume of the cultivation area 21 can be regulated. By sliding the partition 18 towards the midpoint of the reservoir, as shown on FIG. 3, the space previously occupied by water (aka the cultivation area 21) can be reduced and the space occupied by the bed material configured for water treatment can be increased, accordingly. By sliding the partition back, the space occupied by the bioreactor (area 31) is reduced and the space occupied by the cultivation area 21 is increased. The bed material can be easily added to / removed from the water treatment area prior to or after adjusting the volume thereof.

It may be preferred that upon adjusting the volume of the water treatment area 31 (that involves modifying the volume of solid particles therewithin), the water and air supply into the facility is halted. In some instances, provision of the water treatment area 31 within the facility and/or regulating the volume occupied by bed material may require adjustment of the water flow through the distribution piping 11A, 11B and/or redistribution of the elements 11A, 11B, 13 within the water circulation and aeration system 10.

Alternatively, the partition 18 may be provided as a stationary element.

By shifting the partition 18, the entire facility 100 can be turned into a cultivation tank 21 or a water treatment unit 31.

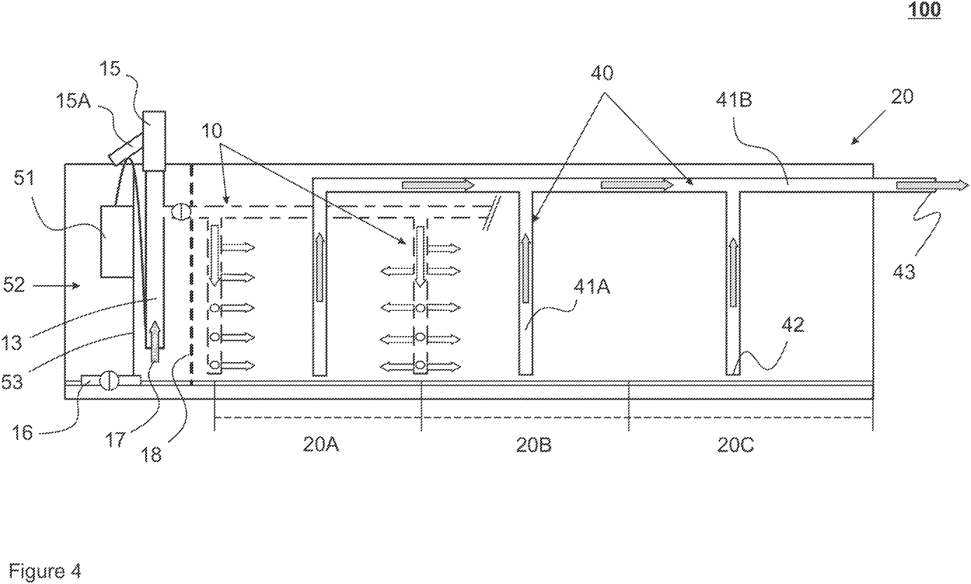

FIG. 4 shows the aquaculture facility 100 according to yet another embodiment. In addition to or alternatively to the water circulation and aeration system 10, the facility 100 illustrated on FIG. 4 comprises a wastewater drain system 40. The wastewater drain system 40 can be thus installed in the reservoir 20 in addition to the water circulation and aeration system 10. Similar to water circulation system 10, the wastewater drain system is realized as an interconnected piping, comprising a number of vertical manifolds 41A configured as vertical pipes each vertical pipe having a (waste)water inlet 42 at its lower end (at the bottom of the reservoir 20). At their upper ends, vertical drain manifolds 41A are interconnected by means of a corresponding interconnecting pipeline 41B provided as an essentially horizontal tube extending along the length of the reservoir 20. The drain system 40 comprises at least one exit 43 configured as a wastewater exhaust pipe, for example, arranged such as to guide waste water outside the reservoir 20 (and the facility 100).

For clarity, the circulation and aeration system 10 is shown on FIG. 4 for a single rotating flow cell 20A in dashed contour lines. It is assumed that taking the present disclosure as a whole, a skilled person would have no difficulty in extrapolating provision of the system 10 to the rest of the reservoir 20.

The wastewater drain system 41 is designed and arranged in the reservoir 20 such, as to position each vertical drain manifold 41A substantially in the middle of each cell 20A, 20B, 20C, i.e. at the positions of central drains 14 (FIGS. 1-3). When the wastewater drain system 40 is installed in the tank 20, provision of central drains 14 can be omitted. Vertical drain manifolds 41A thus replace drain apertures 14 located at the bottom of the reservoir (and the sub-terrain infrastructure underlying said drain apertures, such as wells, piping, etc.).

In presented configuration, vertical drain manifolds 41A have lower ends (42) slightly (5-50 mm) above the bottom of the tank 20. The horizontal pipeline 41B may be arranged above or below the water line. Whether the horizontal pipeline 41B is submerged in water, pressure difference causes water flow in the pipeline. In case the horizontal pipeline 41B is raised above the water surface, vertical manifolds 41A may serve as airlift pumps. Additionally or alternatively, depending on configuration, wastewater flow can be aided by conventional water pumps.

Wastewater is typically withdrawn from a MCR facility through drains arranged in the middle of each rotating flow cell. This water carries settling solids, such as uneaten food, fish faeces, bacterial biomass etc. Some water is further removed with the foam formed upon flotation.

Fresh water should be introduced into an aquaculture facility operating according to RAS principle at an amount of about 250 - 2 500 liter per kg of feed consumed. For a facility operating according to PRAS principle, water requirement is about 5 000 - 15 000 liter per kg of feed consumed. Naturally, substantially the same amount of water is removed from a rearing tank as wastewater (excluding water consumed during fish rearing and bound to fish biomass, and evaporated water). In traditional flow-through aquaculture, water demand is more than 50 000 liter per kg of feed consumed.

Wastewater removal is typically implemented through drains (14, FIGS. 1-3) provided as apertures in the bottom a rearing tank connected to subterranean wells and/or a common drainage system. In some instances, provision of infrastructure (wells, drainage system and associated piping) turns out to be laborious and expensive. In some further instances, it may be desired to preserve integrity of a (transport) container used as a rearing tank; therefore, drilling holes in the bottom of said container should be avoided.

The vertical drain system 40 provides a simple and cost-effective solution for the above mentioned cases. Manifolds 41A, 41B can be assembled and installed into the reservoir 20 on-site. Wastewater is drawn (via apertures 42) upwards the vertical drain tubes 41A by suction, for example, from where wastewater is guided along the horizontal pipe 41B towards the exhaust end 43. Drain systems 40 provided in a number of tanks 20 can be further connected, by their exhaust ends 43 to a common drainage system (not shown).

Vertical wastewater drain system 40 enables a rearing tank based aquaculture facility solution implemented without perforations at its bottom. Provision of underground wells and/or drainage piping can be omitted, accordingly. The entire structure is thus rendered more simple, mobile and cost-effectiveness.

The vertical drain system 40 can be used as an independent drainage arrangement (without the system 10) in any kind of appropriate aquaculture facility not limited to the one described in present specification.

In some configurations, the facility 100 further comprises means (not shown) for supplying hydrogen peroxide (H₂O₂) into the reservoir 20. Said means can be configured as a number of valves interconnected with the manifold piping 11A, 11B and/or the airlift pumps(s) 13 configured to supply hydrogen peroxide from appropriate storage tanks into the reservoir 20 through hoses, pipes or similar appliances. Said valves can be optionally automated or semiautomated. Additionally or alternatively, hydrogen peroxide may be injected directly into the cultivation tank 20 optionally mixed with new (inflowing) water.

By directing hydrogen peroxide into water, continuously, semicontinuously or in single (batch) doses/injections, growth of bacterial biofilm on submerged surfaces and/or growth of microorganisms in aqueous phase within the cultivation tank can be effectively prevented. Hence, injection of hydrogen peroxide serves cleaning and disinfection purposes and it can be used to prevent the diseases from spreading (by disinfecting influent water, for example) and/or to treat fish- or other aquatic species cultivated in the tank 20 in case of infection.

Keeping the aeration- and (liquid) flow circulation systems in aquaculture facilities free of biofilm formation is important to ensure a flawless operation of the facility. When microbial growth (on the submerged surfaces and in liquid phase) is successfully prevented, then the pipelines are not blocked, which, in turn, enables preservation of liquid flow/circulation and gas exchange at required levels without manual cleaning of the tank / the circulation system(s) (the latter naturally requires emptying the cultivation tanks and optionally halting down the entire facility). In an absence of bacterial growth on the surfaces and in the aqueous phase, biological oxygen demand (BOD) (aka the amount of oxygen consumed by bacteria and other microorganisms) of the aquaculture system also remains low.

That continuous supply of hydrogen peroxide into the cultivation tank 20 prevents formation of biofilm on the submerged surfaces, as well as prevents microbial growth in the liquid phase, has been confirmed in experiments involving rearing of rainbow trout (Oncorhynchus mykiss).

When hydrogen peroxide is supplied into the cultivation tank 20 continuously, adjusting concentration of H₂O₂ in said tank to a level of about 1 mg/L or below is generally sufficient. Concentration of hydrogen peroxide in the tank (in conditions of continuous supply) is affected by at least the following factors: an amount of new (influent) water directed into the tank (an extent of dilution), an amount of nutritious substances, such as feed substances, present in the tank (mentioned feed substances represent the organic matter that promotes decomposition of hydrogen peroxide), and the cultivated species (which have different toxicity threshold towards hydrogen peroxide). Experimental trials have demonstrated that supplying hydrogen peroxide into the tank 20 at an amount of 3 mg per a liter of inflowing water resulted in concentration of free hydrogen peroxide in said tank being slightly below 1 mg/L. Overall, safe concentration of H₂O₂ in the tank 20 can be achieved through directing hydrogen peroxide thereinto at a continuous rate within a range of 3-5 mg/L.

Moreover, supply of hydrogen peroxide into the tank 20 in single, sufficiently high doses (concentration of H₂O₂ in water up to 100 mg/L) can be used to increase oxygen content in water without endangering the health of aquatic species cultivated in the tank, since hydrogen peroxide degrades relatively rapidly in presence of organic material. Therefore, H₂O₂ can be utilized as an emergency oxidizing agent (in case of a power failure, for example).

Also when supplying H₂O₂ in single doses, its (maximum) concentration depends on both water quality and cultivated species. Experimental trials have demonstrated that hydrogen peroxide supplied in a single dose of 20 mg/L was sufficient to produce oxygen for cultivated fish (such as rainbow trout, for example) for about an hour in an absence of other aeration (half-life of hydrogen peroxide was approximately 15 min). Also in treatment of fish diseases, about 60 min long exposures to H₂O₂ in high concentrations (at about 100 mg/L) are common.

In some instances, hydrogen peroxide is added into the cultivation tank 20 on a semicontinuous basis. In such an event, H₂O₂ is supplied continuously only during predetermined periods, such as during feeding time, for example. Semicontinuous supply of the oxidizing agent (H₂O₂) would thus introduce oxygen into the system during the time periods when oxygen consumption is at its highest.

Still further, by addition of hydrogen peroxide into the aquaculture system, formation of off-flavour metabolites, in particular, geosmin and 2-methylisoborneol (MIB) produced by a range of bacteria and causing taste and odor problems in aqueous cultivation media can be prevented or significantly reduced. Recent research-scale experiments (not shown) have demonstrated effectiveness of H₂O₂ in reducing concentrations of geosmin and MIB in aquaculture facilities (100) equipped with the water circulation and aeration system 10. In view of regulating the content of off-flavour metabolites in water, hydrogen peroxide has proved more effective or as effective as the more aggressive oxidants, such as ozone and a combination of ozone with hydrogen peroxide.

By connecting a number of facilities 100 together and/or to a common water source and/or a draining system, a modular plant for cultivating aquatic species can be constructed, in where each facility 100 / the reservoir 20 constitutes an individual module. In various configurations, such plant may combine a number of modules 100 comprising both the cultivation area 21 and the water treatment area 31 with the modules configured only as cultivation tanks 21 and/or the modules configured only as water treatment units 31. Additionally or alternatively, configurations that combine a number of modules configured as cultivation tanks 21 with at least one module configured as a water treatment unit 31 can be conceived.

The modules 100 can be configured, individually or collectively, to operate in any one of a flow-through mode, a Recirculation Aquaculture System (RAS) mode or a Partial Reuse Aquaculture System (PRAS) mode.

The flow-through mode or PRAS mode (with water flowing through the module(s) without being recirculated or being at least partially recirculated, accordingly) may be beneficial for quarantining the newly arrived fish (the module then serves a quarantine tank) or for “freshening” the fish intended for sale. Modules operating in PRAS mode enable a so called “all-in, all-out” cultivation strategy, where each cultivation module is limited to e.g. a single generation of fish. Thereafter, the unit is cleaned / disinfected. Modules that operate in RAS mode are beneficial in conditions of limited fresh water supplies and/or when a cost-effective water temperature control is required.

By varying interconnections between the modules 100 / reservoirs 20 and/or connections of said modules to the water source(s) and the draining system, each individual module or a number of modules can be configured to operate in any one of these modes and to switch between said modes as required.

By way of example, a module 100 can act as an independent PRAS unit. In such a case the module may comprise the cultivation area 21 and the water treatment area 31. A number of such modules (e.g. 4-6 modules) optionally without the water treatment area 31 can be further connected to a module provided as a water treatment unit (31) to yield a RAS plant. PRAS utilizes about 10 times less water than flow-through systems; whereas a RAS plant provided with at least one water treatment unit utilizes about 1-2% of a total amount of water typically needed for operating a flow-through plant.

The invention further concerns a method for water circulation and aeration in the aquaculture facility 100 implemented according to the embodiments described hereinabove. The method comprises injecting aerated water into the reservoir 20, configured for cultivating aquatic species, via the exhaust ports 12 arranged on a number of vertical manifolds 11A distributed along the length of the reservoir 20 through the essentially horizontal interconnecting piping 11B, whereby a number of adjacent rotating liquid flow patterns is generated such that each said pattern forms an individual rotating flow cell 20A, 20B, 20C. In the method the aerated water is supplied into the manifolds 11A via at least one airlift pump 13. Injection of aerated water into the tank is thus air-driven, i.e. mediated by said airlift pump(s).

Still further, the invention concerns a method for treating water in the aquaculture facility 100 implemented according to the embodiments described hereinabove. In the method, the aquaculture facility 100 comprises the elongated reservoir 20 with the cultivation area 21 for cultivating aquatic species and the water treatment area 31 separated from the cultivation area 21 by the water permeable partition 18. The water treatment area 31 contains bed material configured for any one of mechanical, (bio)chemical or biological processing as described above. In the method, the liquid arriving into the water treatment area 31 from the cultivation area 21 through the partition 18 undergoes purification and/or treatment, wherein purified liquid may penetrate back into the cultivation area 21. Additionally or alternatively, the purified liquid may be withdrawn from the facility. In the method, the volume of the water treatment area 31 and the volume of the cultivation area 21 are regulated by moving the partition 18 along the length of the reservoir 20.

In some configurations, the method further comprises supplying hydrogen peroxide into the reservoir 20, continuously, semiconsciously, or in single doses, in order to prevent biofilm growth and to ensure cleaning and disinfection of influent water and/or water in the cultivation area. Additionally or alternatively, through supplying hydrogen peroxide into the cultivation tank (20), additional or emergency oxygenation of water can be implemented.

The facility 100 is advantageously utilized for cultivating aquatic species. The facility can be utilized for harvesting fish and/or crustaceans (e.g. shrimps, crabs, lobsters, etc).

Fish species suitable for harvesting include, but are not limited to freshwater- and brackish water species, such as Atlantic salmon (Salmo salar), Tilapia species, carps (Cyprinidae), catfish, and the like; and coldwater species, such as rainbow trout (Oncorhynchus mykiss), European whitefish (Coregonus lavaretus), perch (Perca spp.), pikeperch (Sander lucioperca), sturgeon (Acipenser spp.), Arctic char (Salvelinus alpinus), and the like. For those skilled in the art it is clear that the recirculation aquaculture facility 100 can be adapted for harvesting a variety of fish species, ranging from rearing valuable/difficult-to-harvest fish species at relatively small volumes to harvesting large amounts of fish for mass-market and/or for transplanting into natural environment.

In supplementary or alternative embodiments the aquaculture facility 100 can be used for culturing aquatic species other than fish and crustaceans, such as mollusks (e.g. oysters, mussels), amphibians or aquatic reptiles.

Consequently, a skilled person may on the basis of this disclosure and general knowledge apply the provided teachings in order to implement the scope of the present invention as defined by the appended claims in each particular use case with necessary modifications, deletions and additions.

### REFERENCES

1. Watten et al. Hydraulic characteristics of a rectangular mixed-cell rearing unit. Aquacultural Engineering 24 (2000) 59-73.

1. A water circulation and aeration system for an aquaculture facility comprising an elongated reservoir for cultivating aquatic species, which system comprises a number of vertical manifolds distributed along the length of the reservoir through an interconnecting piping and arranged to generate a number of adjacent rotating liquid flow patterns by injecting aerated water into said reservoir via exhaust ports, wherein each said pattern forms an individual rotating flow cell , and wherein the system further comprises at least one airlift pump configured to aerate water and to supply aerated water into the manifolds.

2. The system of claim 1, wherein the manifolds are arranged to generate rotating flow patterns independently for each individual flow cell.

3. The system of claim 1, wherein the flow patterns are established cell-wise in an absence of physical boundaries between the cells.

4. The system of claim 1, wherein the adjacent cells are established by the counter-flow patterns.

5. The system of claim 1, further comprising a foam collector device connected to the airlift pump conduit.

6. An aquaculture facility comprising an elongated reservoir configured to receive liquid to accommodate aquatic species and to establish a cultivation area, and a water circulation and aeration system as defined in claim 1.

7. The facility of claim 6, wherein the reservoir is formed by an enclosed environment on land.

8. The facility of claim 6, wherein the reservoir is an elongated container.

9. The facility of claim 6, further comprising a water treatment area separated from the cultivation area by a partition, which enables water exchange between the cultivation area and the water treatment area.

10. The facility of claim 6, wherein the partition is configured movable along the length of the reservoir, and wherein by moving said partition the volume of the water treatment area and the volume of the cultivation area is regulated.

11. The facility of claim 6, wherein the water treatment area is a compartment filled with bed material configured for purification and/or treatment of liquid arriving thereto from the cultivation area.

12. The facility of claim 6, wherein the water treatment area is configured for nitrification reactions.

13. The facility of claim 6, further comprising a wastewater drain system which comprises a number of vertical manifolds, each said manifold being arranged substantially in the center of a corresponding rotating flow cell, said manifolds being connected at their upper ends through an interconnecting piping configured to withdraw wastewater outside the facility.

14. The facility of claim 6, further comprising means for supplying hydrogen peroxide into the reservoir.

15. The facility of claim 6, configured as a recirculation aquaculture facility .

16. A modular plant for cultivating aquatic species comprising a number of modules, each module being configured as an aquaculture facility according to what is defined in claim 6, wherein said modules are configured to operate, individually or collectively, in any one of a flow-through mode, a Recirculation Aquaculture System mode or a Partial Reuse Aquaculture System mode, and to switch between said modes as required.

17. A method for water circulation and aeration in an aquaculture facility that comprises an elongated reservoir for cultivating aquatic species, in which method aerated water is injected into said reservoir via a number of vertical manifolds distributed along the length of the reservoir through an interconnecting piping, whereby a number of adjacent rotating liquid flow patterns is generated such that each said pattern forms an individual rotating flow cell, in which method aerated water is supplied into the manifolds via at least one airlift pump.

18. A method for treating water in an aquaculture facility according to what is defined in claim 6, which facility comprises an elongated reservoir with a cultivation area for cultivating aquatic species and a water treatment area separated from the cultivation area by a partition, in which method liquid arriving into the water treatment area from the cultivation area through the partition undergoes purification and/or treatment, and in which method the volume of the water treatment area and the volume of the cultivation area are regulated by moving the partition along the length of the reservoir.

19. A method for cultivating aquatic species, comprising providing the aquaculture facility of claim 6, and utilizing the aquaculture facility to cultivate aquatic species.

20. The system of claim 2, wherein the flow patterns are established cell-wise in an absence of physical boundaries between the cells.
